(19) United States
(12) Patent Application Publication
(10) Pub. No.: US 2025/0366766 A1
(43) Pub. Date: Dec. 4, 2025

(54) BRAIN WAVE PATTERN CHARACTERIZATION

(71) Applicant: The Texas A&M University System, College Station, TX (US)

(72) Inventors: James E. Hubbard, JR., College Station, TX (US); Tristan D. Griffith, Albuquerque, NM (US); Kevin Fuentes, Houston, TX (US); Mark J. Balas, College Station, TX (US)

(21) Appl. No.: 19/227,121

(22) Filed: Jun. 3, 2025

Related U.S. Application Data
(60) Provisional application No. 63/655,111, filed on Jun. 3, 2024.

(51) Int. Cl. A61B 5/369; A61B 5/00; A61B 5/16; G16H 50/70

(52) U.S. Cl. CPC A61B 5/369; A61B 5/165; A61B 5/7264; G16H 50/70

(57) ABSTRACT

Methods, systems, and apparatus, including computer programs encoded on a computer storage medium, for characterizing brain wave patterns are disclosed. In one aspect, a method includes the actions of receiving, from first electroencephalogram (EEG) sensors that are physically detecting activity of a brain of a first patient, first EEG sensor outputs. The actions further include, based on the first EEG sensor outputs, determining relationships between the first EEG sensor outputs and first cognitive states of the first patient. The actions further include receiving, from second EEG sensors that are physically detecting activity of a brain of a second patient, second EEG sensor outputs. The actions further include, based on the relationships between the first EEG sensor outputs and the first cognitive states of the first patient and based on the second EEG sensor outputs, determining a cognitive state of the second patient.

### CROSS-REFERENCE TO RELATED APPLICATION

This application claims benefit of U.S. provisional patent application Ser. No. 63/655,111 filed Jun. 3, 2024 and entitled “Systems and Methods for Characterizing Brain Wave Patterns,” which is hereby incorporated herein by reference in its entirety for all purposes.

### STATEMENT REGARDING FEDERALLY SPONSORED RESEARCH OR DEVELOPMENT

Not applicable

### BACKGROUND

Electroencephalography (EEG) is a method to record an electrogram of the spontaneous electrical activity of the brain. The bio signals detected by EEG may represent the postsynaptic potentials of pyramidal neurons in the neocortex and allocortex. The EEG electrodes may be placed along the scalp. Clinical interpretation of EEG recordings may be performed by visual inspection of the tracing or quantitative EEG analysis.

### SUMMARY

An innovative aspect of the subject matter described in this specification may be implemented in a method for characterizing brain wave patterns. The method includes the action of receiving, from first electroencephalogram (EEG) sensors that are physically detecting activity of a brain of a first patient, first EEG sensor outputs. The method includes the action of, based on the first EEG sensor outputs, determining relationships between the first EEG sensor outputs and first cognitive states of the first patient. The method includes the action of receiving, from second EEG sensors that are physically detecting activity of a brain of a second patient, second EEG sensor outputs. The method includes the action of, based on the relationships between the first EEG sensor outputs and the first cognitive states of the first patient and based on the second EEG sensor outputs, determining a cognitive state of the second patient.

Other implementations of this aspect include corresponding systems, apparatus, and computer programs recorded on computer storage devices, each configured to perform the operations of the method.

Embodiments described herein comprise a combination of features and characteristics intended to address various shortcomings associated with certain prior devices, systems, and methods. The foregoing has outlined rather broadly the features and technical characteristics of the disclosed embodiments in order that the detailed description that follows may be better understood. The various characteristics and features described above, as well as others, will be readily apparent to those skilled in the art upon reading the following detailed description, and by referring to the accompanying drawings. It should be appreciated that the conception and the specific embodiments disclosed may be readily utilized as a basis for modifying or designing other structures for carrying out the same purposes as the disclosed embodiments. It should also be realized that such equivalent constructions do not depart from the spirit and scope of the principles disclosed herein.

### BRIEF DESCRIPTION OF THE DRAWINGS

For a more complete understanding of the present disclosure, reference is now made to the following brief description, taken in connection with the accompanying drawings and detailed description, wherein like reference numerals represent like parts.

FIG. 1 illustrates an example system that is configured to determine a cognitive state of a patient based on output from EEG sensors.

FIG. 2 is a flowchart of an example process for determining a cognitive state of a patient based on output from EEG sensors.

FIG. 3 is a flowchart of an example process for generating a dynamic image of brain activity using an array of EEG sensors.

### DETAILED DESCRIPTION

The following discussion is directed to various exemplary embodiments. However, one skilled in the art will understand that the examples disclosed herein have broad application, and that the discussion of any embodiment is meant only to be exemplary of that embodiment, and not intended to suggest that the scope of the disclosure, including the claims, is limited to that embodiment.

Certain terms are used throughout the following description and claims to refer to particular features or components. As one skilled in the art will appreciate, different persons may refer to the same feature or component by different names. This document does not intend to distinguish between components or features that differ in name but not function. The drawing figures are not necessarily to scale. Certain features and components herein may be shown exaggerated in scale or in somewhat schematic form and some details of conventional elements may not be shown in interest of clarity and conciseness.

Unless the context dictates the contrary, all ranges set forth herein should be interpreted as being inclusive of their endpoints, and open-ended ranges should be interpreted to include only commercially practical values. Similarly, all lists of values should be considered as inclusive of intermediate values unless the context indicates the contrary. Where numerical ranges or limitations are expressly stated, such express ranges or limitations should be understood to include iterative ranges or limitations of like magnitude falling within the expressly stated ranges or limitations (e.g., from about 1 to about 10 includes, 2, 3, 4, etc.; greater than 0.10 includes 0.11, 0.12, 0.13, etc.).

In the following discussion and in the claims, the terms “including” and “comprising” are used in an open-ended fashion, and thus should be interpreted to mean “including, but not limited to . . . .” Use of the term “optionally” with respect to any element of a claim is intended to mean that the subject element is required, or alternatively, is not required. Both alternatives are intended to be within the scope of the claim. The term “couple” or “couples” is intended to mean either an indirect or direct connection. As used herein, the terms “approximately,” “about,” “substantially,” and the like mean within 10% (i.e., plus or minus 10%) of the recited value. Thus, for example, a recited angle of “about 80 degrees” refers to an angle ranging from 72 degrees to 88 degrees.

Noninvasive multi-channel electroencephalographic (EEG) systems may be used in clinics to record brain electrical activities and detect, diagnose, and treat traumatic brain injuries, epilepsy, tumors, cerebral palsy, strokes, and other neurological diseases. In addition to the stand-alone EEG devices often used by hospital professionals, portable wireless EEG devices exhibit stronger market demands as they can be used in home care settings. While these units may involve many channels of high temporal resolution data, EEG devices do not currently process the inherent high spatial resolution data needed for true brain wave imaging. This is because of the limited capabilities of the software platforms currently employed. This limitation has long plagued and greatly limited the use of such devices in the presence of a rising demand for true noninvasive brain wave imaging. The reason is that EEG signals are complex, stochastic, and nonlinear, and their measures can be impacted by many unknown intrinsic and extrinsic system-related or user-related parameters. Thus, new computational tools are needed to analyze these signals in order to achieve high classification accuracy. To circumvent these barriers, this technology supports the research and commercialization activities needed to create advanced and innovative EEG analysis software to yield a service that can generate modern noninvasive brain wave imaging in the form of a brain mapping model produced in real-time and with high fidelity.

The technology described in this specification makes use of advanced EEG analysis algorithms and software with high temporal resolutions, allowing for real-time measurements of brain activity with millisecond precision and high spatial resolution by providing a modal representation and mapping of brain waves that captures activity across the entire brain volume. It employs output-only system identification algorithms to estimate a canonical state-space model of neural dynamics occurring in space and time. The resulting mapping is a reduced-order modal decomposition that enables high spatial resolution, noninvasive brain wave imaging in near real-time using EEG. The method also employs modern state estimation to develop brain wave mapping using a minimal number of EEG channels. In addition, this state space architecture can continuously update an adaptive modal brain wave model by estimating an exogenous input to the brain wave system. This continuous update is adaptive because the embedded state estimator monitors its performance and can modify its parameters by a closed-loop action. In this manner, the adaptive algorithm accounts for any nonlinearities and stochasticity impacting the EEG measures. The estimator is robust to general process uncertainty in the identified plant dynamics and thus can reconstruct the EEG measures in real time and with high accuracy and fidelity. The result is a software platform that provides a powerful tool combined with EEG biomarkers for understanding brain function and dysfunction at a relatively low cost and with minimal risk to research participants or patients. The technology can be applied across the entire spectrum of commercially available EEG systems that include low density and high density electrodes to yield high spatial and temporal brain wave maps.

In some examples, EEG technology may not produce brain wave images. Instead, the user is provided with many channels of high temporal resolution streaming data that requires a trained specialist to interpret. Also, these EEG devices do not currently process and present the inherent high spatial resolution data needed for true brain wave imaging, diagnosis and prognosis. This is because of the limited capabilities of the software platforms in some cases. This limitation may limit the use of such devices in the presence of a rising demand for true noninvasive brain wave imaging. The reason is that EEG signals are complex, stochastic, and nonlinear, and their measures can be impacted by many unknown intrinsic and extrinsic system or user related parameters. Techniques like qEEG may not generate true brain wave patterns, but instead, they may post process EEG data to give a mapping of the power spectra of the data. Some example means of mapping brain waves is expensive, time consuming and uncomfortable for the patient and involves the use of large, expensive, and complex magnetic resonance imaging (MRI), functional magnetic resonance imaging (fMRI), positron emission tomography (PET) machines and the like. While these large expensive devices may provide images, they are static, averaged, non-real-time and contain no cognitive system dynamics.

Non-invasive brain imaging methods may be used to study the brain and may provide researchers and clinicians with tools for understanding brain function and dysfunction in health and disease. Non-invasive brain wave imaging may be used because it allows medical professionals to study the brains structure without invasive techniques that require penetrating the skull or injecting substances into the brain.

This specification describes a high-level EEG data processing method which overcomes these limitations by providing high spatial resolutions mappings of the brain and providing robustness to many artifacts. The method uses an adaptive output-only system identification and state estimation to determine a state space model, in near real time, of brain waves and has several exciting features. First, the technology described in this specification offers a way to achieve high spatial and temporal resolution mappings with a sparse sensor network, which is a significant advantage over some example non-invasive brain imaging techniques. Second, the technology achieves this at relatively low cost, using wireless data transmission and near realtime imaging without the patient discomfort and expense inherent in the use of fMRI, MRI, and PET machines.

Third, because the method results in a high-fidelity model, it can be used with various model-based machine learning and artificial intelligence (AI) techniques for the classification many brain disorders including seizures and concussions. Fourth, while some example techniques such as ElectroCochleoGraphy (ECOG), near-infrared spectroscopy (NIRS), magnetoencephalography (MEG), and quantitative electroencephalography (qEEG) may also offer the advantage of high temporal and spatial resolution, but they may not be able to achieve the same level of resolution with a sparse sensor network and in near real time. In summary, the technology described in this specification offers the significant advantages for brain wave imaging that include (a) low cost, (b) non-invasiveness, (c) versatility, (d) accessibility, (e) near real-time imaging, and (f) minimum risk to research participants or patients.

This technology presents a modeling approach for analyzing spatio-temporal brain wave dynamics in near real time. The nonlinear, nonstationary behavior of brain wave measures and general uncertainty associated with the brain makes it difficult to apply modern system identification techniques to such systems. The algorithms address the issue of modeling and imaging brain waves and biomarkers generally, treating the nonlinear and nonstationary dynamics in near real-time. Using our unique and novel modal state-space formulation, leads to intuitive, physically significant models which are used for analysis and diagnosis.

The technology may utilize “black box” output only system identification techniques to the biomarker of electroencephalogram data. The result is a linear time invariant model that admits brain wave modes of human cognitive states. The weak non-linearities and stochasticity present in the data are accommodated using an custom adaptive feedback scheme that adjust the model in near real-time to yield less than one percent error with the concomitant data stream. The resulting imaged brain wave “modes” may be linearly independent and can be likened to the keys on a piano. Weighted linear combinations can produce “chords” which can be combined to produce music and reveal the “symphony of the brain.” This modal state-space formulation leads to intuitive, physically significant models which has broad applicability for analysis, classification and diagnosis. These space-time modal patterns of the brain have the potential for widespread application in the diagnosis of Alzheimer's disease, dementia, seizures, human emotions, concussion protocols and the like.

There are other example non-invasive techniques. A first example technique is electroencephalography (EEG), which is a method of recording electrical activity in the brain. Electrodes are attached to the scalp to measure the electrical activity of the brain. This technique may be used in clinical settings for the diagnosis and treatment of brain disorders. A second example technique is magnetoencephalography (MEG), which may be similar to EEG in that it employs an array of sensors on the scalp but measures the magnetic fields of the brain. A third example technique is functional magnetic resonance imaging (fMRI), which uses magnetic fields and radio waves to measure changes in blood flow in the brain. This method can be used to create images of the brain function. A fourth example technique is near infrared spectroscopy (NIRS), which uses lights to measure blood flow and oxygenation levels in the brain and is typically used in studies of children and young infants. A fifth example technique is positron emission tomography (PET).

While each of these methods have different advantages and disadvantages, EEG has some notable advantages over most of these techniques which are more expensive both in terms of equipment, time, patient comfort and the expertise needed to operate them. MRI, fMRI and PET scans may take a longer time to perform, which limits their use in emergency or critical care settings. While they may provide spatial resolution, they have limited temporal resolution. EEG has temporal resolution which makes it a good candidate for studying the dynamics of neural processing and rapid changes in the brain. In some examples, EEG has limited spatial resolution and has high sensitivity to noise and artifacts such as eye movements, muscle activity and environmental factors. The technology described in this specification, when paired with example EEG systems, addresses these problems.

The technology described in this specification generates high resolution brain wave imaging using a sparse network sensor array as opposed to 32, 64, and/or 128 sensor arrays. While other techniques which can also include ECOG, NIRS, MEG, and hdEEG also offer advantages in terms of temporal and spatial resolution, they may not be able to achieve the same level of resolution with a sparse sensor network. These example techniques may require a larger number of sensors to achieve the high spatial resolution that the approach described in this specification can, and may not be able to achieve the same level of temporal resolution as the techniques described in this specification using state estimation and observability.

The technology described in this specification offers unique advantages in terms of achieving high spatial and temporal resolution with a sparse sensor network, which could be useful in applications where a large number of sensors is not feasible or desirable, such as in wearable or portable brain imaging devices.

The technology described in this specification uses output-only system identification to determine a state space model using EEG has several advantages over other example non-invasive brain imaging methods. The first advantage is high temporal and spatial resolution. The technique offers both high temporal resolution, allowing for real-time measurements of brain activity with millisecond precision, and high spatial resolution, providing a modal representation of brain waves that captures activity across the entire brain volume. The second advantage is non-invasiveness. The technique is non-invasive, meaning it does not require penetrating the skull or injecting substances into the brain, which reduces the risk of harm to research participants or patients.

The third advantage is low cost. The technique uses relatively inexpensive and available EEG equipment, making it more cost-effective than other non-invasive brain imaging methods, such as fMRI and PET. The fourth advantage is versatility. The technique can be used to study a wide range of brain functions, including basic sensory processing, cognition, and motor control, as well as to diagnose and monitor brain disorders. The fifth advantage is accessibility. The technique can be performed by researchers and clinicians with varying levels of expertise and does not require specialized training in advanced imaging techniques. Overall, the technique offers a unique approach to non-invasive brain imaging, providing a powerful tool for understanding brain function and dysfunction with high temporal and spatial resolution, at a relatively low cost and with minimal risk to research participants or patients.

The human brain remains one of the most complex and least understood systems in science. Breakthroughs in understanding cognitive dynamics, mental health, and neuroplasticity depend on tools that can resolve brain activity in real-time and at high resolution. The Adaptive Digital Twin (ADT) is a technology platform that models and visualizes brain wave activity in real-time using physics-based statistical representations. ADT employs a dynamic systems approach using operational modal analysis (OMA) to extract eigenvalues and eigenvectors that reveal the real-time structure of cognition. This technology enables the detection of subtle transitions in mental state, emotion, and cognitive load, positioning ADT as useful in neuroscience, education, and healthcare.

The ADT platform is built on the concept of using system identification to generate a dynamic state-space model from EEG signals. The technique leverages operational modal analysis to extract dominant modes of brain activity in the form of eigenvalues and modal shapes. These are continuously updated using an adaptive observer-based scheme that accommodates nonstationary and nonlinear features in EEG data. In some implementations, these updates may occur without requiring extensive pre-processing or machine learning. The result is a real-time cognitive map, which may be considered a dynamic twin of brain function that can be visualized and interpreted. This representation has applications in monitoring attention, detecting emotional shifts, identifying cognitive fatigue, and tracking recovery after neural injury. Because the method is computationally lightweight, it is deployable in portable, wireless EEG systems and other systems where power usage is a concern. The integration of cloud-based access and Health Insurance Portability and Accountability Act (HIPAA) compliant storage further enhances the clinical and research scalability of ADT.

ADT may be used for subject identification. ADT may be used to successfully identify individual EEG signatures using modal patterns, which utilizes its ability to extract personalized cognitive fingerprints. In subject identification, dominant eigenvalues and associated mode shapes may be unique and consistent across sessions for different subjects. The technology offers potential for biometric identification, especially in secure environments where physiological authentication is needed. This application may be used for access control, where traditional biometrics such as fingerprints identification and iris scanning may be inadequate.

ADT may be used for emotion classification using valence and arousal. ADT may be applied to segment EEG data and used to compute Hamiltonian eigenvalues from the adapted system matrix. These eigenvalues provide discriminative features corresponding to states of high/low valence and arousal. Compared to example techniques such as event-related potential (ERP) and frequency band analyses, ADT showed improved temporal resolution and interpretability. This capability may enable real-time emotional state tracking in educational environments, therapeutic sessions, and user-experience testing.

ADT may be used for stroke onset detection that may be via signal complexity. The adaptive update scheme that is included in ADT enables it to track sudden changes in brain wave dynamics. The onset of stroke symptoms may be preceded by identifiable shifts in system eigenvalues and increased signal complexity. In this case, ADT may be used for continuous neurological monitoring and early stroke detection. The non-invasive, real-time capability of ADT may support wearable monitoring systems that issue alerts before critical events, especially in elderly or high-risk populations.

ADT may be used for high-resolution mapping with sparse sensor networks. ADT may infer high-resolution cognitive maps from sparse EEG sensor arrays. By initializing a baseline model and continuously adapting it using partial observations, ADT may reconstruct full-brain or near-full brain modal behavior from as few as eight sensors. This capability may reduce hardware constraints and may open pathways to cost-effective, scalable neurotechnology. It holds promise for applications in classrooms, remote clinics, and mobile diagnostics where full EEG systems are impractical.

ADT may be used for real-time or near real-time mental health monitoring. As mental health challenges rise, especially in the context of digital media exposure, the need for non-invasive, real-time monitoring tools has grown. ADT provides a framework for assessing cognitive and emotional state dynamics in naturalistic settings. By mapping changes in dominant eigenvalues and modal structures from EEG data, ADT enables the detection of transient emotional responses, attentional lapses, and cognitive overload in real time. This capability may be relevant for monitoring individuals during tasks such as social media browsing or emotionally charged video consumption. ADT may analyze EEG data collected from patients exposed to varying digital stimuli. The goal may be to identify dynamic biomarkers such as eigenvalue trajectories and increases in modal complexity that correspond to shifts in mental state. This application may be used to develop “digital phenotyping” tools that can translate raw neurophysiological signals into interpretable indicators of mental health status. By providing continuous feedback on internal cognitive state without the need for active user input, ADT may be used for early detection of anxiety, depressive rumination, or attentional dysregulation.

The ADT mode features may be interpreted to various cognitive states. A first example feature and/or mode parameter may be frequency. A high frequency, such as thirty to fifty hertz, may relate to conscious attention, perceptual binding, and/or memory encoding. A very high frequency, such as above sixty hertz, may relate to hyperarousal, stress, and/or sensory overload. A second example feature and/or mode parameter may be damping. A low damping, such as below ten percent, may relate to sustained focus and/or prolonged cognitive engagement. A high damping, such as above twenty percent, may relate to transient attention and/or disrupted processing. A third example feature and/or mode parameter may be complexity. A moderate complexity, such as ten to twenty percent, may relate to emotional-cognitive integration and/or evaluative thought. A high complexity, such as above thirty percent, may relate to ambiguous affective state, conflict resolution, and/or decision tension. A fourth example feature and/or mode parameter may be traveling wave direction. Waves moving from the frontal area of the brain to the parietal area of the brain may relate to top-down cognitive control and/or executive processing. Waves moving from the occipital area of the brain to the frontal area of the brain may relate to sensory-driven processing, vigilance, and/or alertness. A fifth example feature and/or mode parameter may be standing waves/localized mode that may relate to local processing, reflexive, and/or autonomic state. A sixth example feature and/or mode parameter may be stable eigenvalue trajectory (over trials) that may relate to cognitive stability and/or habitual or rehearsed thinking. A seventh example feature and/or mode parameter may be a sudden shift in mode shape or frequency that may relate to cognitive transition, novelty detection, and/or surprise.

The above description summarizes features derived from ADT modal decomposition and their interpretation in terms of cognitive states. Each mode parameter, such as frequency, damping ratio, complexity, and spatial mode behavior, is linked to an inferred mental state. For instance, higher frequencies are associated with active attention or stress, while damping ratios differentiate between sustained engagement and transient focus. Mode complexity captures the integration of affective and cognitive dynamics, and spatial patterns, such as traveling or standing waves, reveal hierarchical versus local processing. Longitudinal features like stable eigenvalue trajectories indicate habituation, while abrupt changes suggest novelty or cognitive transition. These relationships provide a conceptual framework for interpreting brain activity in real-time using the ADT platform. The model decomposition may be displayed as a series of modal mappings, each associated with an intrinsic frequency.

ADT includes a graphical user interface that visualizes modal amplitudes, phase interactions, and dominant brain dynamics in a 3D scalp map. These visualizations allow users, for example, neuroscientists, clinicians, and educators, to observe the temporal evolution of cognitive states and transitions. The system can be integrated with machine learning models that classify these patterns into discrete behavioral or emotional categories, making it a hybrid platform for both discovery science and applied monitoring.

The ADT architecture may include various modules. The modules may include a signal acquisition layer that interfaces with EEG systems, a preprocessing and filtering layer that includes bandpass filtering and noise removal features, and a model estimation engine that include real-time modal analysis features. The modules may also include an adaptive observer that may include a feature to update the state with partial data, a visualization and export layer that plots modal energy and eigenvalue evolution, and a cloud integration layer that provides secure data access and sharing.

In addition to these modules, an additional innovation in the ADT platform is the interpretability of the system matrix eigenvalues derived from EEG data. These eigenvalues represent the natural frequencies and damping ratios of underlying neural dynamics. For instance, eigenvalues near the imaginary axis in the complex plane often correspond to sustained oscillatory behavior, which may be linked to attentional processes or cognitive engagement. In contrast, eigenvalues with larger real parts (more negative) may indicate rapidly decaying activity, which may signify transient or reactive mental states. During real-time processing, ADT tracks the evolution of these eigenvalues to monitor shifts in cognitive load, emotional valence, or mental fatigue. Specific eigenvalue clusters may emerge consistently during transitions between rest, focused attention, and affective processing. By animating these trajectories over time, the system may reveal temporal modes of brain function, providing a high-resolution, physics-based window into cognition. These insights may be integrated into the visualization module, where users can observe not only scalp-level maps of modal energy but also the eigenvalue spectra evolving across time. This dual-domain representation bridges the gap between signal dynamics and cognitive state interpretation, offering both spatial and temporal understanding of brain activity.

The ADT platform may analyze EEG data from users engages in social media activity and may include real-time neural effects of digital engagement. Using this data, the ADT platform may model cognitive state transitions, tracking dominant eigenvalue trajectories, complexity metrics, and modal structures in response to emotionally provocative or attentionally demanding stimuli. In some implementations, this may identify dynamic biomarkers that reflect moment-to-moment changes in mental state and generate a foundation for various diagnostic tools and interventions.

The ADT platform may also be used for clinical and educational. Clinically, it can aid in diagnosis and intervention for disorders such as depression, post-traumatic stress disorder (PTSD), attention-deficit/hyperactivity disorder (ADHD), and mild cognitive impairment. In education, it may provide tools to understand attention and learning states in real time and inform personalized education plans. It may enable cognitive readiness monitoring for high-performance operators, such as pilots, air traffic controllers, and/or drone controllers. Further, by enabling real-time brain state interpretation, ADT empowers mental health professionals with lower-cost tools. The use of sparse sensor arrays and cloud-based visualization platforms opens up possibilities for use in schools and mobile clinics. ADT may contribute a structured mathematical framework to brain state decoding. By grounding its analysis in control theory and dynamical systems, it offers an alternative to data-driven black-box models, enhancing transparency and reproducibility in neuroscience research. It provides avenues for correlating biophysical signals with functional states, moving from coarse averages to temporally localized state representations. The platform also sets the stage for ethical, privacy-conscious mental state monitoring. With real-time estimation capabilities and minimal preprocessing, ADT avoids the need for invasive procedures or large-scale data collection, making it suitable for deployment in diverse global contexts, such as from rural clinics and mobile education labs.

ADT is a diagnostic tool and a platform that transforms modeling, understanding, and interacting with the brain. By converting raw EEG signals into dynamic cognitive maps, ADT can serve as the core of intelligent systems that respond to human thought in real time, enabling adaptive tutoring platforms, early mental health interventions, and neuroadaptive machines.

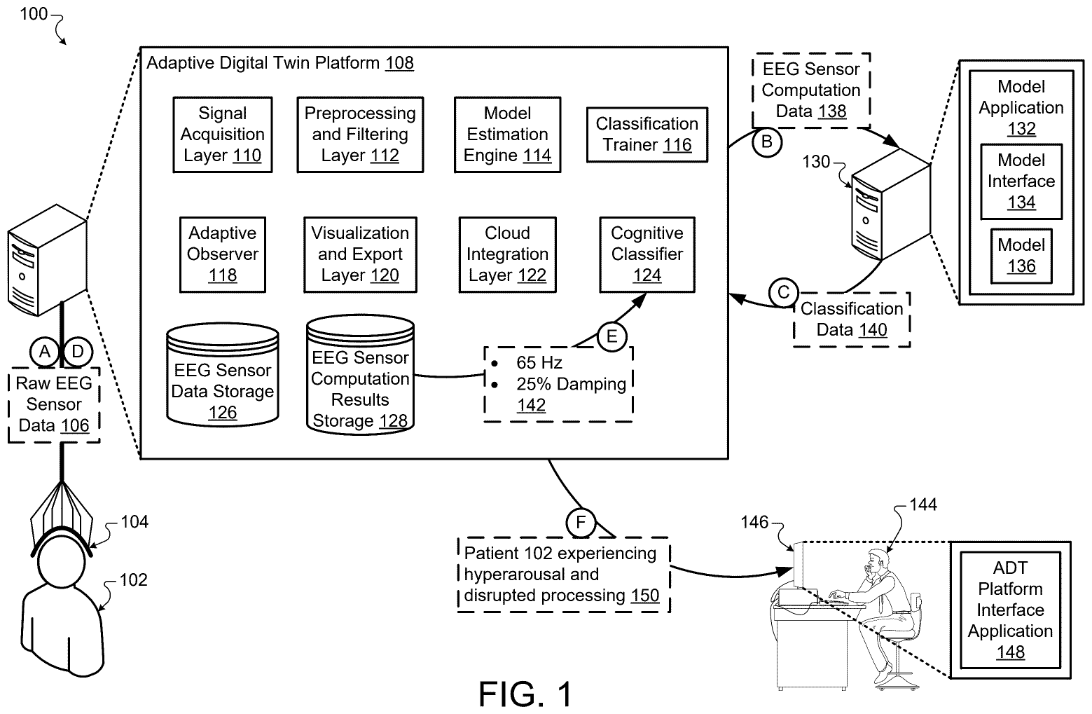

FIG. 1 illustrates an example system 100 that is configured to determine a cognitive state of a patient based on output from EEG sensors. Briefly, and as described in more detail below, the system 100 includes an adaptive digital twin platform 108 that is receives and analyzes output from EEG sensors 104 connected to the patient 102 and/or other patients. The adaptive digital twin platform 108 generates a cognitive classifier 124 that is configured to classify the cognitive state of a patient based on an analysis of the outputs of EEG sensors. The classification may be based on various numerical values that the system calculates based on the outputs of the EEG sensors. FIG. 1 includes various stages A through F that may illustrate the performance of actions and/or the movement of data between various components of the system 100 and/or between the system 100 and other devices. The system 100 may perform these stages in any order.

In more detail, the user 144 may be analyzing the brain function of the patient 102. The user 144 may be a clinician and/or another type of individual who may be interested in the brain function of the patient 102. The patient 102 may be connected to various EEG sensors 104 that are attached to the head of the patient 102. The number of EEG sensors 104 may vary. For example, the number may be sixty-four, one-hundred twenty-eight, ten, and/or any other number of sensors. The EEG sensors 104 may be attached to a cap that the patient 102 wears over the head of the patient 102. Each EEG sensor 104 may detect electrical activity of the brain of the patient 102 at the location of each EEG sensor 104. The adaptive digital twin platform 108 may analyze the output of the EEG sensors 104 to determine the cognitive state of the patient 102.

In advance of determining the cognitive state of the patient 102, the adaptive digital twin platform 108 may generate or update the cognitive classifier 124. By generating or updating the cognitive classifier 124, the adaptive digital twin platform 108 may be able to classify the cognitive state of a given patient based on an analysis of the outputs of EEG sensors. The discussion below describing the generation or updating of the cognitive classifier 124 will use the raw EEG sensor data 106 from the EEG sensors 104 connected to the patient 102. However, the raw EEG sensor data may be collected from any EEG sensors that are connected to any patient including the EEG sensors 104 connected to the patient 102. The raw EEG sensor data may be collected from multiple patients that are connected to the same or different EEG sensors. The number of EEG sensors connected to each patient may also be different.

To generate or update the cognitive classifier 124 and in stage A, the adaptive digital twin platform 108 may receive the raw EEG sensor data 106 from the EEG sensors 104 connected to the patient 102. The raw EEG sensor data 106 may be collected over a period of time, such as twenty minutes, over one or more sessions. The adaptive digital twin platform 108 may include a signal acquisition layer 110. The signal acquisition layer 110 may be configured to interface with the EEG sensors 104. The signal acquisition layer 110 may store the raw EEG sensor data 106 collected from the EEG sensors 104 in the EEG sensor data storage 126. The signal acquisition layer 110 may store additional metadata such as timestamps, data identifying a particular EEG sensor, data identifying a particular EEG sensor array, data identifying the patient 102 with additional safeguards to protect patient privacy, demographic data related to the patient 102, and/or any other similar metadata. The demographic data may include the age, race, gender, occupation, ethnicity, marital status, income level, education, and/or any other similar demographic data. In some implementations, the metadata may include medical history of the patient 102.

The adaptive digital twin platform 108 may include a preprocessing and filtering layer 112. The preprocessing and filtering layer 112 may include one or more bandpass filters. The preprocessing and filtering layer 112 may also be configured to remove noise from the raw EEG sensor data 106. In some implementations, the preprocessing and filtering layer 112 may process the raw EEG sensor data 106 before or after storing the raw EEG sensor data 106 in the EEG sensor data storage 126.

The adaptive digital twin platform 108 may include a model estimation engine 114. The model estimation engine 114 may be configured to analyze the processed and filtered EEG sensor data. The model estimation engine 114 may generate eigenvalues and eigenvectors from the processed and filtered EEG sensor data. The model estimation engine 114 may operate in real-time as the EEG sensors 104 generate sensor data and as the signal acquisition layer 110 and preprocessing and filtering layer 112 receive and process the raw EEG sensor data 106. The model estimation engine 114 may store the eigenvalues, eigenvectors, and/or other calculations in the EEG sensor computation results storage 128.

The adaptive digital twin platform 108 may include an adaptive observer 118. The adaptive observer 118 may be configured to accommodate nonstationary and nonlinear features in raw EEG sensor data 106 and/or the processed and filtered EEG sensor data. In some implementations, the adaptive observer 118 may accommodate nonstationary and nonlinear features in raw EEG sensor data 106 and/or the processed and filtered EEG sensor data without requiring preprocessing and/or machine learning. The adaptive observer 118 may store and calculated data in the EEG sensor computation results storage 128.

The adaptive digital twin platform 108 may include a visualization and export layer 120. The visualization and export layer 120 may be configured to generate a real-time cognitive map of brain function. This real-time cognitive map can be viewed and interpreted by a user 144. The visualization and export layer 120 may also plot modal energy and eigenvalue evolution.

The adaptive digital twin platform 108 may include a cloud integration layer 122. The cloud integration layer 122 may be configured to provide secure data access and data sharing. The cloud integration layer 122 may allow users, such as user 144 to view and interact with the visualizations generated by the visualization and export layer 120.

The adaptive digital twin platform 108 may include a classification trainer 116. The classification trainer 116 may be configured to generate or update the cognitive classifier 124. The cognitive classifier 124 may be configured to determine a cognitive state of the patient 102 based on the analysis of the raw EEG sensor data 106.

In stage B, the classification trainer 116 may interact with the computing device 130. The computing device 130 may be any type of computing device that is capable of communicating with other computing devices. For example, the computing device 130 may be a desktop computer, laptop computer, server, mobile phone, wearable device, tablet, and/or any other similar type of electronic device. The computing device 130 may include a model application 132 that includes a model interface 134 and a model 136.

The classification trainer 116 may provide EEG sensor computation data 138 to the model application 132. In some implementations, the classification trainer 116 may select particular EEG sensor computation data 138 from the EEG sensor computation results storage 128. For example, the classification trainer 116 may select EEG sensor computation data 138 that related to a certain demographic, such as females between the ages of thirty and forty. In some implementations, the classification trainer 116 may select the EEG sensor computation data 138 without specifying any demographic filter. In some implementations, the classification trainer 116 may select the EEG sensor computation data 138 that originated from a specific number of EEG sensors in the cap attached to the patient, such as sixty-four sensors. In some implementations, the classification trainer 116 may select the EEG sensor computation data 138 that originated from a specific cap that was attached to one or more patients.

The model interface 134 of the model application 132 may receive the EEG sensor computation data 138. The model interface 134 may provide the EEG sensor computation data 138 to the model 136. In some implementations, the model 136 may be large language model or any other type of artificial intelligence model. The model 136 may be trained using machine learning.

In some implementations, the classification trainer 116 may generate instructions to include with the EEG sensor computation data 138. The instructions may be based on the type of classifications the classification trainer 116 is attempting to determine. For example, the instructions may be to determine a likely cognitive state that corresponds to different eigenvalues, eigenvectors, and/or changes in the eigenvalues and/or eigenvectors. The instructions may include current classifications used by the cognitive classifier 124 as may be the case if the classification trainer 116 it attempting to update the cognitive classifier 124.

In stage C, the model 136 may analyze the EEG sensor computation data 138 and generate the classification data 140. The model application 132 may provide the classification data 140 to the classification trainer 116. The classification trainer 116 may implement or update the cognitive classifier 124. The classification data 140 may specify characteristics of the data in the EEG sensor computation results storage 128 and correlate those characteristics with various cognitive states. The characteristics of the data in the EEG sensor computation results storage 128 may relate to the characteristics of waves moving through the brain. The visualization and export layer 120 may generate a visualization of these waves moving through the brain. For example, frequencies as represented by the eigenvalues may related to conscious attention, perceptual binding, or memory encoding for high frequencies such as between thirty and fifty hertz. Very high frequencies such as above sixty hertz may be related to hyperarousal, stress, or sensory overload.

As another example, the damping values of the waves may be related to sustained focus or prolonged cognitive engagement for damping values that are less than ten percent. Damping values such as those that are greater than twenty percent may be related to transient attention or disruptive processing. Other characteristics such as the complexity of the waves, the direction of the waves, the mode of the waves, the stability of the eigenvalues, and/or a sudden shift in the mode, shape, or frequencies of the waves may have related cognitive states as discussed above. In some implementations, the complexity of the waves may be based on the frequencies that make up the waves. For example, waves with more frequency components may have a higher complexity percentage than waves with fewer frequency components.

With the cognitive classifier 124 updated based on the classification data 140 and in stage D, the adaptive digital twin platform 108 may be configured to collect raw EEG sensor data 106 and determine the likely cognitive state of the patient 102. The adaptive digital twin platform 108 may receive the raw EEG sensor data 106 from the EEG sensors 104. The signal acquisition layer 110, the preprocessing and filtering layer 112, the model estimation engine 114, the adaptive observer 118, and/or the visualization and export layer 120 may process the raw EEG sensor data 106 using the techniques described above. The model estimate engine 114 and/or the adaptive observer 118 may generate numerical values based on the preprocessed and filtered raw EEG sensor data 106 and store those numerical values in the EEG sensor computation results storage 128.

In stage E, the cognitive classifier 124 accesses the data in the EEG sensor computation results storage 128. In some implementations, the cognitive classifier 124 may access the data in the EEG sensor computation results storage 128 in response to a request from the user 144. The cognitive classifier 124 may compare the frequency, damping, complexity, wave direction, mode, trajectory, mode shift, and/or any other similar value to the various thresholds and ranges in the cognitive classifier 124. For example, the cognitive classifier 124 may receive a frequency value of sixty-five hertz and a damping value of twenty five percent. Based on the frequency value of sixty-five hertz, the cognitive classifier 124 may determine that the patient 102 is experiencing hyperarousal. Based on the damping value of twenty five percent, the cognitive classifier 124 may determine that the patient 102 is experiencing disrupted processing.

In stage F, the cognitive classifier 124 generates a cognitive results packet 150 that includes the results generated by the cognitive classifier 124. In this example, the cognitive results packet 150 indicates that the patient 102 is experiencing disrupted processing and is experiencing hyperarousal. The adaptive digital twin platform 108 provides the cognitive results packet 150 to the computing device 146. The computing device 146 may execute an adaptive digital twin platform interface application 148 that is configured to communicate with the adaptive digital twin platform 108. The user 144 may view the results included in the cognitive results packet 150 on the computing device 146. The computing device 146 may be any type of computing device that is capable of communicating with other computing devices. For example, the computing device 146 may be a desktop computer, laptop computer, server, mobile phone, wearable device, tablet, and/or any other similar type of electronic device.

Any of the adaptive digital twin platform 108, the computing device 130, and/or the computing device 146 may be any type of computing device that is configured to transmit to and receive data from other computing devices. The devices may communicate with other computing devices using a wide area network, a local area network, the internet, a wired connection, a wireless connection, and/or any other type of network or connection. The wireless connections may include Wi-Fi, short-range radio, infrared, and/or any other wireless connection. Some of the components, interfaces, modules, and/or layers of the devices may be implemented in a single computing device or distributed over multiple computing devices. Some of the components, interfaces, modules, and/or layers may be in the form of virtual machines or software containers that are hosted in a cloud in communication with disaggregated storage devices. Some of the components, interfaces, modules, and/or layers may be implemented by one or more processors through the execution of computer-executable instructions stored in memory.

The devices may include a communication interface, one or more processors, memory, and hardware. The communication interface may include communication components that enable the systems to transmit data and receive data from devices connected to the wireless carrier network. The communication interface may include an interface that is configured to communicate with base stations of a wireless carrier network. The communication interface may receive data that other devices transmit to the base stations and/or transmit data to the base stations for transmission to the other devices. In some implementations, the communication interface may be configured to communicate over a wide area network, a local area network, the internet, a wired connection, a wireless connection, and/or any other type of network or connection. The wireless connections may include Wi-Fi, short-range radio, infrared, and/or any other wireless connection.

The hardware may include user interface, data communication, or data storage hardware. For example, the user interfaces may include a data output device (e.g., visual display, audio speakers), and one or more data input devices. The data input devices may include, but are not limited to, combinations of one or more of keypads, keyboards, mouse devices, touch screens that accept gestures, microphones, voice or speech recognition devices, and any other suitable devices.

The memory may be implemented using computer-readable media, such as computer storage media. Computer-readable media includes, at least, two types of computer-readable media, namely computer storage media and communications media. Computer storage media includes volatile and non-volatile, removable and non-removable media implemented in any method or technology for storage of information such as computer-readable instructions, data structures, program modules, or other data. Computer storage media includes, but is not limited to, RAM, ROM, EEPROM, flash memory or other memory technology, CD-ROM, digital versatile disks (DVD), high-definition multimedia/data storage disks, or other optical storage, magnetic cassettes, magnetic tape, magnetic disk storage or other magnetic storage devices, or any other non-transmission medium that can be used to store information for access by a computing device. In contrast, communication media may embody computer-readable instructions, data structures, program modules, or other data in a modulated data signal, such as a carrier wave, or other transmission mechanism. In some implementations, the data stored in the memory may be stored externally from the systems.

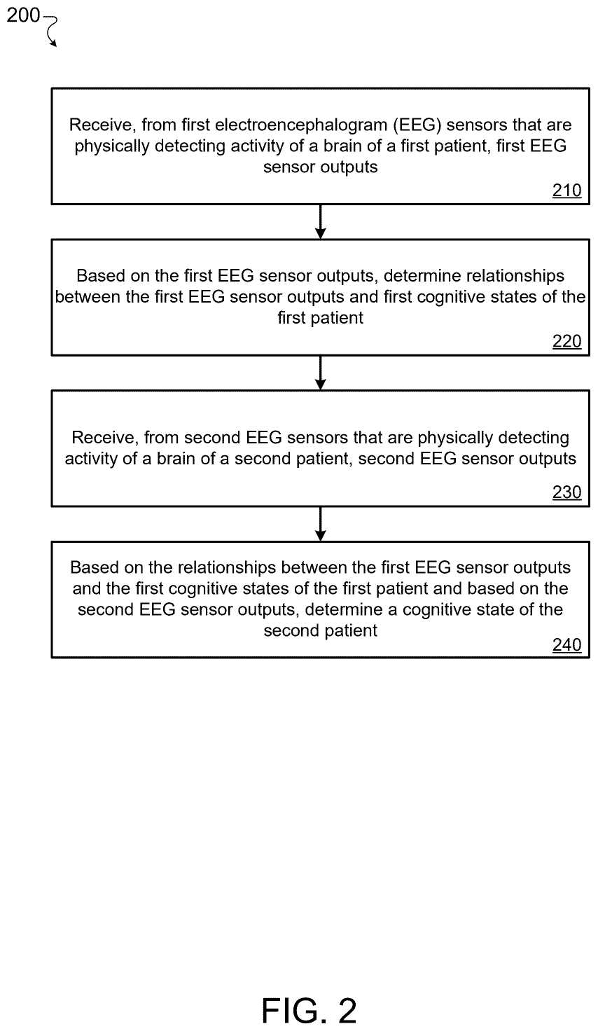

FIG. 2 is a flowchart of an example process 200 for determining a cognitive state of a patient based on output from EEG sensors. The process 200 receives EEG sensor output from EEG sensors connected to a patient. Based on the EEG sensor outputs, the process 200 determines relationships between the EEG sensor outputs and various cognitive states of the patient. The process 200 receives additional EEG sensor outputs and uses the relationships to determine a cognitive state of the patient connected to the EEG sensors that generated the additional EEG sensor outputs. The process 200 will be described by being performed by the system 100 and will include references to other components in FIG. 1. The process 200 may be performed by a single computing device, which may be a virtual device, and/or split across multiple computing devices that may include virtual devices. In some implementations, the process 200 may be performed by an application.

The adaptive digital twin platform 108 receives, from first electroencephalogram (EEG) sensors that are physically detecting activity of a brain of a first patient, first EEG sensor outputs (210). The adaptive digital twin platform 108 may continuously collect the first EEG sensor outputs from the first EEG sensors. The adaptive digital twin platform 108 may preprocess and filter the first EEG sensor outputs.

The adaptive digital twin platform 108, based on the first EEG sensor outputs, determines relationships between the first EEG sensor outputs and first cognitive states of the first patient (220). In some implementations, the adaptive digital twin platform 108 may determine numerical values based on the first EEG sensor outputs. The adaptive digital twin platform 108 may continuously calculate the numerical values as the adaptive digital twin platform 108 receives additional first EEG sensor outputs. In some implementations, the numerical values are eigenvalues and eigenvectors. In some implementations, the numerical values are frequency values, damping values, and complexity values of waves that are moving through the brain of the first patient. In some implementations, the numerical values may reflect directions of traveling waves in the brain of the first patient.

In some implementations, the adaptive digital twin platform 108 provides the first EEG sensor outputs to a model. The model determines the relationships between the first EEG sensor outputs and first cognitive states of the first patient. In some implementations, the adaptive digital twin platform 108 provides the numerical values to the model. The model determines the relationships between the numerical values and first cognitive states of the first patient. In some implementations, the adaptive digital twin platform 108 provides the model EEG sensor outputs and/or numerical values collected from one or more EEG sensor arrays connected to one or more patients to the model. The model determines relationships between the numerical values and/or the EEG sensor outputs and cognitive states of the one or more patients. In some implementations, the EEG sensor outputs and/or numerical values may be grouped based on patient identity, demographic characteristics, EEG sensor array, and/or any other similar category.

In some implementations, the model may be a large language model. In some implementations, the model may be part of the adaptive digital twin platform 108. In some implementations, the model and/or the training process used by the model may leverage supervised learning, unsupervised learning, semi-supervised learning, naive Bayes, Bayesian network, decision trees, artificial neural networks, fuzzy logic models, probabilistic classification models, random forest modeling, k-means cluster, a decision tree algorithm, a support vector machine (SVM) algorithm, an ensemble of trees algorithm, and/or a gradient-boosted trees algorithm.

In some implementations, the relationships between the EEG sensor outputs and cognitive states may be various thresholds and/or ranges. The thresholds and/or ranges may be mapped to various cognitive states. In some implementations, there may be different thresholds and/or ranges for different demographic groups. In some implementations, the relationships may be included in a classifier that is configured to receive numerical values that are based on given EEG sensor outputs and output a given cognitive state of a given patient.

The adaptive digital twin platform 108 receives, from second EEG sensors that are physically detecting activity of a brain of a second patient, second EEG sensor outputs (230). In some implementations, the second patient and the first patient are the same or different people. In some implementations, the first EEG sensor and the second EEG sensors are the same set of EEG sensors.

The adaptive digital twin platform 108, based on the relationships between the first EEG sensor outputs and the first cognitive states of the first patient and based on the second EEG sensor outputs, determines a cognitive state of the second patient (240). In some implementations, the adaptive digital twin platform 108 calculates numerical values based on the second EEG sensor outputs and compares those numerical values to the various thresholds and/or ranges of the relationships between the EEG sensor outputs and cognitive states. Based on the outcome of those comparisons, the adaptive digital twin platform 108 determines a likely cognitive state of the second patient.

In some implementations, the adaptive digital twin platform 108 may update the relationships between the EEG sensor outputs and/or numerical values and the cognitive states. The adaptive digital twin platform 108 may update them based on feedback from a patient and/or a user viewing the results. In some implementations, the adaptive digital twin platform 108 selects the relationships between the EEG sensor outputs and/or numerical values based on characteristics of the patient and/or the EEG sensors. These characteristics may include demographic characteristics of the patient, type of EEG sensors, number of EEG sensors, spacing of EEG sensors, location of EEG sensors relative to body parts of the patient such as ears, eyes, etc., and/or any other characteristic.

In some implementations, the adaptive digital twin platform 108 may determine whether a patient is the same patient who was previously connected to the EEG sensors. In this case, the adaptive digital twin platform 108 may compare EEG sensor outputs and/or numerical values to previous EEG sensor outputs and/or numerical values that may be associated with specific patients. Based on that comparison, the adaptive digital twin platform 108 may determine whether a patient is the same patient who was previously connected to the EEG sensors.

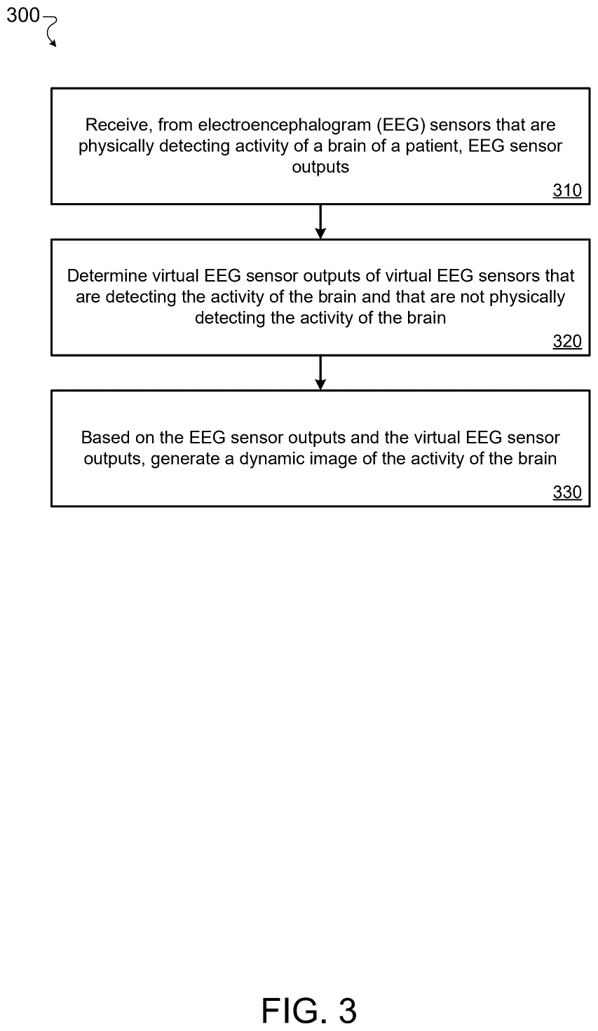

FIG. 3 is a flowchart of an example process 300 for generating a dynamic image of brain activity using an array of EEG sensors. The process 300 receives EEG sensor outputs from EEG sensors connected to a patient. The process 300 estimates additional EEG sensor outputs from virtual EEG sensors that are not physically connected to the patient. Based on the EEG sensor outputs and the additional EEG sensor outputs, the process 300 generates a dynamic image of brain activity of the patient. The process 300 will be described by being performed by the system 100 and will include references to other components in FIG. 1. The process 300 may be performed by a single computing device, which may be a virtual device, and/or split across multiple computing devices that may include virtual devices. In some implementations, the process 300 may be performed by an application.

The adaptive digital twin platform 108 receives, from electroencephalogram (EEG) sensors that are physically detecting activity of a brain of a patient, EEG sensor outputs (310). The adaptive digital twin platform 108 may continuously collect the EEG sensor outputs from the EEG sensors. The adaptive digital twin platform 108 may preprocess and filter the EEG sensor outputs.

The adaptive digital twin platform 108 determines virtual EEG sensor outputs of virtual EEG sensors that are detecting the activity of the brain and that are not physically detecting the activity of the brain (320). The virtual EEG sensor may be EEG sensors that do not actually exist. Instead, the adaptive digital twin platform 108 is configured to estimate an output of additional virtual EEG sensors as if they were actual EEG sensors connected to the patient.

In some implementations, the adaptive digital twin platform 108 provides the EEG sensor outputs to a state estimation model that is configured to determine a likely state of the virtual EEG sensors. In this case, the virtual EEG sensor outputs may be based on the likely state of the virtual EEG sensors. In some implementations, the state estimation model may be selected based on demographic data of the patient that the adaptive digital twin platform 108 determines or accesses. In some implementations, the state estimation model is a generic model that is not associated with demographic data of the patient. In some implementations, the state estimation model based may be selected based on the number of the EEG sensors that the adaptive digital twin platform 108 determines or accesses and a comparison between the number of the EEG sensors and a threshold. The threshold may vary based on the demographic characteristics of the patient.

In some implementations, the virtual EEG sensor outputs may be based on the number of EEG sensors. The virtual EEG sensor outputs may vary based a comparison between the number of EEG sensors and a different, or the same, threshold number of sensors. In some implementations, the virtual EEG sensor outputs may be based on the density of EEG sensors. The virtual EEG sensor outputs may vary based a comparison between the density of EEG sensors and a threshold density.

The adaptive digital twin platform 108, based on the EEG sensor outputs and the virtual EEG sensor outputs, generates a dynamic image of the activity of the brain (330). In some implementations, the dynamic image of the activity of the brain is provided to a diagnosis model and/or classifier that is configured to output a likely diagnosis and/or cognitive state of the patient. In some implementations, the adaptive digital twin platform 108 may receive additional EEG sensor outputs. Based on those additional EEG sensor outputs, the adaptive digital twin platform 108 may update the state estimation model. This feedback may provide a mechanism to update and tune the state estimation model to improve the estimation of the virtual EEG sensor outputs.

While several implementations have been provided in the present disclosure, it should be understood that the disclosed systems and methods may be embodied in many other specific forms without departing from the spirit or scope of the present disclosure. The present examples are to be considered as illustrative and not restrictive, and the intention is not to be limited to the details given herein. For example, the various elements or components may be combined or integrated in another system or certain features may be omitted or not implemented. Accordingly, the scope of protection is not limited to the embodiments described herein, but is only limited by the claims that follow, the scope of which shall include all equivalents of the subject matter of the claims. Unless expressly stated otherwise, the steps in a method claim may be performed in any order. The recitation of identifiers such as (a), (b), (c) or (1), (2), (3) before steps in a method claim are not intended to and do not specify a particular order to the steps, but rather are used to simplify subsequent reference to such steps.

Also, techniques, systems, subsystems, and methods described and illustrated in the various implementations as discrete or separate may be combined or integrated with other systems, modules, techniques, or methods without departing from the scope of the present disclosure. Other items shown or discussed as directly coupled or communicating with each other may be indirectly coupled or communicating through some interface, device, or intermediate component, whether electrically, mechanically, or otherwise. Other examples of changes, substitutions, and alterations are ascertainable by one skilled in the art and could be made without departing from the spirit and scope disclosed herein.

Each and every claim is incorporated into the specification as an aspect of the present disclosure. Thus, the claims are a further description and are an addition to the aspects of embodiments disclosed herein. The discussion of a reference herein is not an admission that it is prior art to the presently disclosed subject matter, especially any reference that may have a publication date after the priority date of this application. The disclosures of all patents, patent applications, and publications cited herein are hereby incorporated by reference, to the extent that they provide exemplary, procedural or other details supplementary to those set forth herein.

What is claimed is:

1. A computer-implemented method, comprising:
receiving, from first electroencephalogram (EEG) sensors that are physically detecting activity of a brain of a first patient, first EEG sensor outputs;
based on the first EEG sensor outputs, determining relationships between the first EEG sensor outputs and first cognitive states of the first patient;
receiving, from second EEG sensors that are physically detecting activity of a brain of a second patient, second EEG sensor outputs; and
based on the relationships between the first EEG sensor outputs and the first cognitive states of the first patient and based on the second EEG sensor outputs, determining a cognitive state of the second patient.

2. The method of claim 1, comprising:
determining numerical values based on the first EEG sensor outputs,
wherein determining relationships between the first EEG sensor outputs and the first cognitive states of the first patient comprises determining relationships between the numerical values and the first cognitive states.

3. The method of claim 2, wherein the numerical values are eigenvalues and eigenvectors.

4. The method of claim 2, wherein the numerical values are frequency values, damping values, and complexity values.

5. The method of claim 1, wherein determining the relationships between the first EEG sensor outputs and the first cognitive states of the first patient comprises providing the first EEG sensor outputs to a model.

6. The method of claim 1, comprising:
generating a classifier that is configured to receive numerical values that are based on given EEG sensor outputs and output a given cognitive state of a given patient.

7. The method of claim 1, wherein:
determining a direction of traveling waves in the brain of the first patient based on the first EEG sensor outputs,
wherein determining relationships between the first EEG sensor outputs and the first cognitive states of the first patient comprises determining relationships between the direction of the traveling waves and the first cognitive states.

8. The method of claim 1, comprising:
based on the first EEG sensor outputs, determining relationships between the first EEG sensor outputs and the first patient;
receiving, from third EEG sensors that are physically detecting activity of a brain of a third patient, third EEG sensor outputs; and
based on the relationships between the first EEG sensor outputs and the first patient and based on the third EEG sensor outputs, determining whether the first patient and the third patient are a same patient.

9. A system, comprising:
one or more processors; and
memory including a plurality of computer-executable components that are executable by the one or more processors to perform acts comprising:
receiving, from first electroencephalogram (EEG) sensors that are physically detecting activity of a brain of a first patient, first EEG sensor outputs;
based on the first EEG sensor outputs, determining relationships between the first EEG sensor outputs and first cognitive states of the first patient;
receiving, from second EEG sensors that are physically detecting activity of a brain of a second patient, second EEG sensor outputs; and
based on the relationships between the first EEG sensor outputs and the first cognitive states of the first patient and based on the second EEG sensor outputs, determining a cognitive state of the second patient.

10. The system of claim 9, wherein the acts comprise:
determining numerical values based on the first EEG sensor outputs,
wherein determining relationships between the first EEG sensor outputs and the first cognitive states of the first patient comprises determining relationships between the numerical values and the first cognitive states.

11. The method of claim 10, wherein the numerical values are eigenvalues and eigenvectors.

12. The method of claim 10, wherein the numerical values are frequency values, damping values, and complexity values.

13. The system of claim 9, wherein determining the relationships between the first EEG sensor outputs and the first cognitive states of the first patient comprises providing the first EEG sensor outputs to a model.

14. The system of claim 9, wherein the acts comprise:
generating a classifier that is configured to receive numerical values that are based on given EEG sensor outputs and output a given cognitive state of a given patient.

15. The system of claim 9, wherein:
determining a direction of traveling waves in the brain of the first patient based on the first EEG sensor outputs,
wherein determining relationships between the first EEG sensor outputs and the first cognitive states of the first patient comprises determining relationships between the direction of the traveling waves and the first cognitive states.

16. The system of claim 9, wherein the acts comprise:
based on the first EEG sensor outputs, determining relationships between the first EEG sensor outputs and the first patient;
receiving, from third EEG sensors that are physically detecting activity of a brain of a third patient, third EEG sensor outputs; and
based on the relationships between the first EEG sensor outputs and the first patient and based on the third EEG sensor outputs, determining whether the first patient and the third patient are a same patient.

17. One or more non-transitory computer-readable media storing computer-executable instructions that upon execution cause one or more processors to perform acts comprising:
receiving, from first electroencephalogram (EEG) sensors that are physically detecting activity of a brain of a first patient, first EEG sensor outputs;
based on the first EEG sensor outputs, determining relationships between the first EEG sensor outputs and first cognitive states of the first patient;
receiving, from second EEG sensors that are physically detecting activity of a brain of a second patient, second EEG sensor outputs; and
based on the relationships between the first EEG sensor outputs and the first cognitive states of the first patient and based on the second EEG sensor outputs, determining a cognitive state of the second patient.

18. The media of claim 17, wherein the acts comprise:
determining numerical values based on the first EEG sensor outputs,
wherein determining relationships between the first EEG sensor outputs and the first cognitive states of the first patient comprises determining relationships between the numerical values and the first cognitive states.

19. The media of claim 17, wherein determining the relationships between the first EEG sensor outputs and the first cognitive states of the first patient comprises providing the first EEG sensor outputs to a model.

20. The media of claim 17, wherein the acts comprise:
generating a classifier that is configured to receive numerical values that are based on given EEG sensor outputs and output a given cognitive state of a given patient.
